(19) United States
(12) Patent Application Publication
(10) Pub. No.: US 2024/0105322 A1
(43) Pub. Date: Mar. 28, 2024

(54) SOFTWARE APPLICATION TO STANDARDIZE ACCESS TO SURGICAL PROCEDURE INVENTORY BENCHMARKS AND DATA COLLECTION AND IMPLEMENTATION OF SURGICAL PROCEDURE BENCHMARKS FOR TRAY RATIONALIZATION AND SUPPLY OPTIMIZATION

(71) Applicants: Benjamin Chadwick Wood, Raleigh, NC (US); David Jon Rowe, Raleigh, NC (US); Martyn Knowles, Raleigh, NC (US)

(72) Inventors: Benjamin Chadwick Wood, Raleigh, NC (US); David Jon Rowe, Raleigh, NC (US); Martyn Knowles, Raleigh, NC (US)

(21) Appl. No.: 18/484,153

(22) Filed: Oct. 10, 2023

Related U.S. Application Data
(63) Continuation of application No. PCT/US2022/022803, filed on Mar. 31, 2022.
Application No. PCT/US2022/022803 is a continuation of application No. 17/459,173, filed on Aug. 27, 2021.
(60) Provisional application No. 63/172,378, filed on Apr. 8, 2021.

(51) Int. Cl. G16H 40/20

(52) U.S. Cl. CPC G16H 40/20

(57) ABSTRACT

The subject matter disclosed herein includes a computerized method that supports the process of data collection for analysis and rationalization of instrument trays within a healthcare environment. The method, performed using a processor, includes for receiving instrument usage information related to instrument trays for use in the healthcare environment. Information related to preference card instrumentation, equipment, and disposable articles is stored within a database. A tray configuration is provided for a determined procedure. Instrument usage information is received, where the instrument suage information is related to instrument trays after use in the healthcare environment. It is determined, based on the received instrument usage after use in the healthcare environment, whether articles placed within an instrument tray are being used at a frequency below a predetermined threshold. Preference card information and tray configuration are updated for the determined procedure based on the determination.

### PRIORITY CLAIM

This application is a continuation of PCT Patent Application No. PCT/US2022/022803 filed on Mar. 31, 2022, entitled “SOFTWARE APPLICATION TO STANDARDIZE ACCESS TO SURGICAL PROCEDURE INVENTORY BENCHMARKS AND DATA COLLECTION AND IMPLEMENTATION OF SURGICAL PROCEDURE BENCHMARKS FOR TRAY RATIONALIZATION AND SUPPLY OPTIMIZATION”, which claims priority to U.S. patent application Ser. No. 17/459,173 filed on Aug. 27, 2021, entitled “SOFTWARE APPLICATION TO STANDARDIZE ACCESS TO SURGICAL PROCEDURE INVENTORY BENCHMARKS AND DATA COLLECTION AND IMPLEMENTATION OF SURGICAL PROCEDURE BENCHMARKS FOR TRAY RATIONALIZATION AND SUPPLY OPTIMIZATION,” which claims the benefit of U.S. Provisional Patent Application No. 63/172,378 filed on Apr. 8, 2021, the entire contents of all of which are incorporated by reference herein.

### TECHNICAL FIELD

This disclosure relates to reconfiguration of instrument trays and disposable supplies for surgical procedures. More specifically, it relates to a software application to standardize access to surgical procedure inventory benchmarks and data collection and implementation of surgical procedure benchmarks for tray rationalization and supply optimization.

### BACKGROUND

Surgical instrument trays have a significant number of re-usable instruments that are not used during cases. Various interests, such as hospitals, ambulatory surgery centers, sterile processing companies, medical device manufacturers, software platforms, and consulting firms, desire to minimize instrument tray size. Smaller tray size reduces the time required to clean and assemble trays, breakdown trays, reconfigure trays, and sterilize trays. Smaller tray size results in less time required to set up an operating room and cost savings resulting from a decrease in necessary instrument inventory levels, maintenance, and purchasing of instruments that are not used.

Currently, there are no systems or technology products that perform instrument tray rationalization utilizing empiric usage data collected intraoperatively. Instead, data collection and tray rationalization are performed manually if they are performed at all. Without comprehensive data and tray rationalization utilizing empiric usage data collected intraoperatively, the time and expense associated with managing surgical trays is likely to be suboptimal.

Accordingly, a need exists to reduce the number and range of instruments, instrument trays, and disposable supplies provided for use in surgical procedures in operating rooms to reduce operative expenses, inventory levels, and hospital operating expense.

### SUMMARY

The subject matter disclosed herein includes a computer-implemented method comprising instructions stored on a non-transitory computer-readable storage medium and executed on a computing device provided with a hardware processor and a memory for collection and rationalization of instrument trays within a healthcare environment. The method includes determining and storing: equipment information related to equipment inventory, disposable article information related to disposable article inventory, and preference card information related to surgical equipment and disposable articles used during a procedure. A tray configuration is determined for a procedure based on the preference card information, the equipment information, and the disposable article information, where the tray configuration identifies articles to be placed within an instrument tray. Instrument usage information is determined for articles placed within the instrument tray during the procedure. Based on the instrument usage information, it is determined whether the articles placed within the instrument tray are used at a frequency below a predetermined threshold. In response to determining that the articles placed within the instrument tray are used at a frequency below the predetermined threshold, the preference card information and the tray configuration for the procedure are updated.

### BRIEF DESCRIPTION OF THE DRAWINGS

The present embodiments are illustrated by way of example and are not intended to be limited by the figures of the accompanying drawings. In the drawings:

FIG. 1 is a functional block diagram illustrating an end-to-end process in accordance with at least one embodiment of the present invention.

FIG. 2 is a functional block diagram illustrating a workflow for the identification and data collection capability in accordance with at least one embodiment of the present invention.

FIG. 3 is a functional block diagram illustrating data collection analysis, buffer calculation, and tray configuration in accordance with at least one embodiment of the present invention.

FIG. 4 is a functional block diagram illustrating a machine learning model and instrument recommendation algorithm for tray configurations in accordance with at least one embodiment of the present invention.

FIG. 5 is a functional block diagram illustrating a consolidation engine configured to reduce the number of trays sharing similar instruments and reduce tray instance proliferation in accordance with at least one embodiment of the present invention.

FIG. 6 is a functional block diagram illustrating the use of a web interface for a clinical benchmark database in accordance with at least one embodiment of the present invention.

FIG. 7 is a functional block diagram illustrating a distributed data processing environment suitable for operation of a instrument tray data collection and rationalization program in accordance with at least one embodiment of the present invention.

### DETAILED DESCRIPTION

In contrast to current methods and systems for management and configuration of instrument trays and disposable supplies for surgical procedures which do not perform instrument tray rationalization utilizing empiric usage data collected intraoperatively, the improved methods and systems disclosed herein provide for tray rationalization and supply optimization including standardized access to surgical procedure inventory benchmarks, data collection, and surgical procedure benchmarks. The subject matter disclosed herein includes a toolset and application that supports the collection of data regarding instrument usage on instrument trays for both internal and vendor-specific for surgical procedures. Usage data may be grouped by procedure, preference card, surgeon, surgical specialty, hospital location by tray and instrument per case. Once collected, a rationalization engine utilizes the usage data to configure new trays based on usage and existing tray configurations. An analytics algorithm displays a list of affected preference cards and instrument associations for one or more trays that can be consolidated or eliminated if the proposed trays were to be used. Data association visualizations and statistical analysis provide visualizations which identify the intersection of the proposed tray with other existing associated trays to build consolidated trays. A consolidation engine identifies opportunities to consolidate trays for hospitals to reduce the overall number of trays. The consolidation engine utilizes associations between the preference cards for procedures performed within a hospital and calculates the impact on instrument inventory for the proposed transfer of instruments to consolidated trays. Preference cards are then updated to create a closed feedback loop regarding instruments listed on the preference cards for procedure trays.

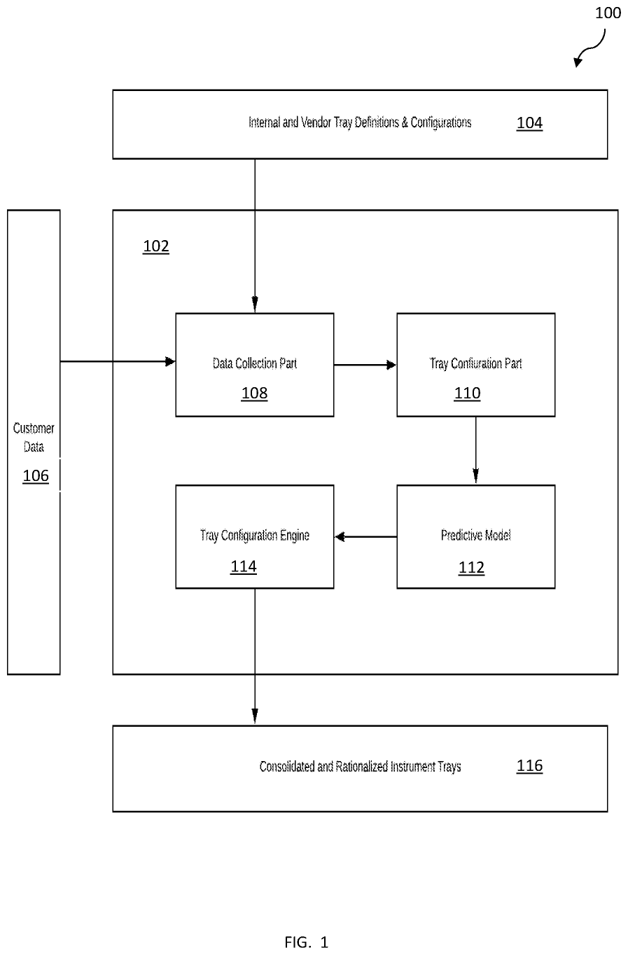

The present invention is a method, computer program product, and system for data collection and rationalization of instrument trays within a healthcare environment. FIG. 1 is a functional block diagram illustrating an end-to-end process in accordance with at least one embodiment of the present invention. Referring to FIG. 1, process 100 may be performed by instrument tray data collection and rationalization program 102. Process 100 may begin when internal and vendor tray definitions and configurations 104 and customer data 106 are received by data collection part 108. Once data is collected, tray configuration part 110 may determine one or more tray configurations based on the data.

Next, predictive model 112 identifies specific instruments to be added and a quantity needed of those instruments. In addition to using instruments that have actual usage collected in counts, predictive model 112 may also use related instruments and tray associations to determine which instruments are likely needed. This determination may be based on, for example, specialty, location, surgeon, procedure, and CPT code(s).

Next, tray configuration engine 114 determines proposed tray configurations and presents to the user which existing preference cards or pick lists utilize the proposed tray. This presentation of the proposed tray configurations helps the user understand the scope of the change within the hospital. Tray configuration engine 114 may also generate and display a mathematical percentage of overlap between the newly proposed tray and an existing tray.

Finally, consolidated and rationalized instrument trays 116 are determined. The proposed consolidated and rationalized instrument trays 116 may combine and reduce multiple existing trays into fewer proposed trays.

The following benefits may be associated with the methods and systems disclosed herein: reduced surgical case duration, reduced sterile processing costs, reduced purchase of instruments, reduced need for repair of instruments not used on trays, reduction in inventory of instruments, reduction in overall internal supply chain costs, reduction in facility operational expense, reduction in turnover time between surgical cases, reduction in setup time in the operating room, improved accuracy of instrument usage resulting in reduction of excess instruments, and improved hospital inventory planning. Additional details of the present invention are described in greater detail below with reference to the Figures.

### Tray Collection

A web application may perform collection of instrument usage during a surgical procedure. The collection of instrument usage information may be associated with or grouped by instrument tray, surgeon, procedure, Current Procedural Terminology (CPT) code(s), surgical specialty, operating room, personnel assignments.

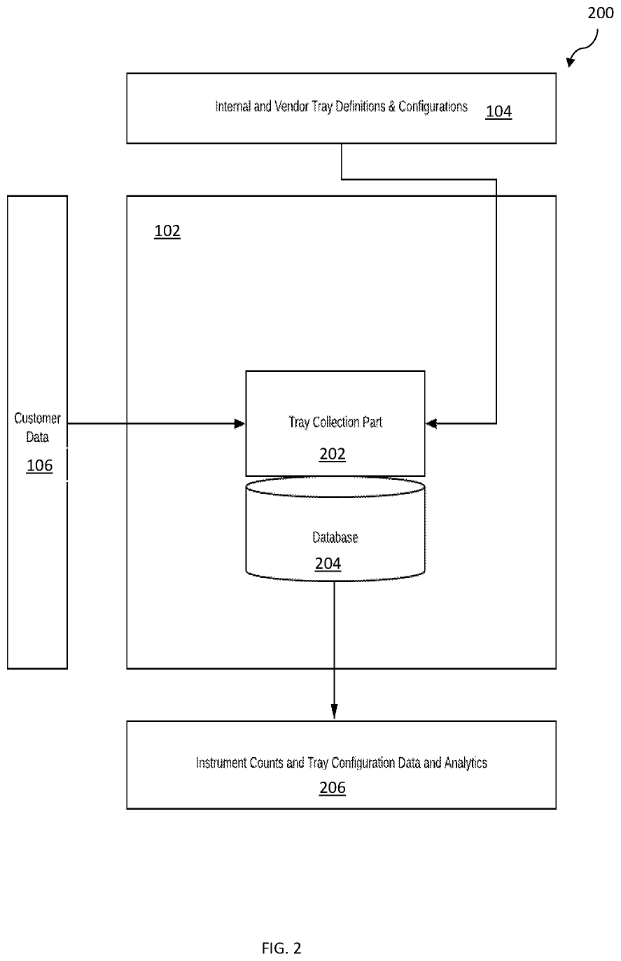

FIG. 2 is a functional block diagram illustrating an exemplary workflow for the identification and data collection capability in accordance with at least one embodiment of the present invention. Referring to FIG. 2, process 200 begins when tray collection part 202 receives internal and vendor tray definitions and configurations 104 and customer data 106. Tray collection part 202 may be associated with, and communicatively coupled to, database 204 for storing data. The output of process 200 includes instrument counts and tray configuration data and analytics 206.

The collection process includes generating and/or sending an automated notification to a user signaling the user to count the required trays at the assigned time perioperatively. Quantification of the number of times each procedure has been counted is used to flag or highlight cases that need to be counted. Additionally, guidance on cases targeted for data collection may be prioritized by Specialty, Location, Surgeon, Procedure and/or CPT code to ensure adequate sample size and count distribution across the aforementioned variables. Collected data is integrated into the software to allow collection of counts at a detailed level. Data visibility allows a user of the software to identify data usage for existing trays and create a newly proposed tray configuration. This enables users to see existing tray configurations including the average usage per instrument by Procedure, Surgeon, Location, Preference Card (or Pick List) and the number of counts performed for that tray. Those data allow a user to select commonly used instruments to build a new tray.

### Tray Configuration

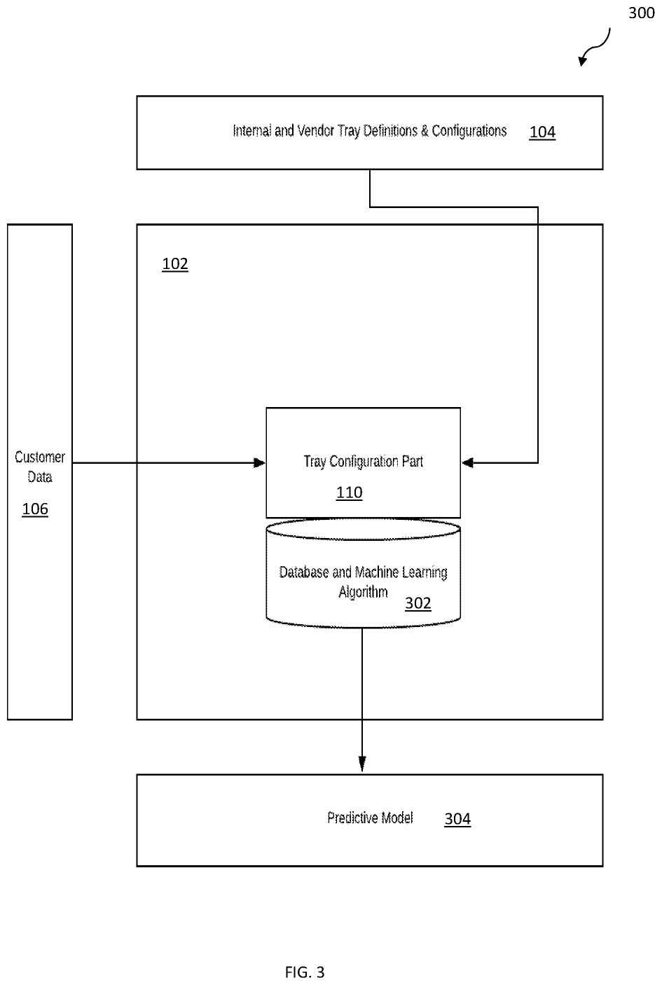

FIG. 3 is a functional block diagram illustrating data collection analysis, buffer calculation, and tray configuration in accordance with at least one embodiment of the present invention. Referring to FIG. 3, process 300 begins when tray configuration part 110 receives internal and vendor tray definitions and configurations 104 and customer data 106.

Tray configuration part 110 may be associated with, and communicatively coupled to, database and machine learning algorithm 302. For example, database and machine learning algorithm 302 may generate proposed tray configurations and data-based associations allowing the user to see which existing preference cards or pick lists utilize the proposed tray to understand the scope of the change within the hospital. The tool generates and displays the mathematical percentage of overlap for the newly proposed tray and the existing tray on the preference card or pick list to determine if replacing the tray on the cards is feasible. Iterations of the data can be conducted until a one hundred percent overlap is achieved for a tray associated with the proposed tray allowing the user to replace the existing tray with a new tray. Benchmark data for corresponding tray and supply usage and recommendations are exposed within the tool to allow creation of proposed trays based on benchmarks to reduce amount of sample required.

A buffering algorithm predicts tray configuration based on past usage and other approved tray configurations. In one embodiment, the buffering algorithm utilizes machine learning. The buffering algorithm uses benchmark data that has comparable usage for the same surgical procedure that has been rationalized and standardized within the benchmarking engine.

The buffering algorithm is extended to interact with signaling algorithms to provide the capability to maintain optimal configurations based on actual usage and buffering predictions. Buffering depends on the benchmark engine to predict which instrument or supply is commonly needed above a specific usage-based metric.

Audit process configurations may be used to validate and modify preference cards, for example, based on perioperative feedback. The new preference card configurations and recommended changes to preference cards include individual or low velocity peel pack items identified by the rationalization engine to complete the process.

Web service access to the benchmarks allows applications outside of the proprietary collection tool. For example, a benchmark database storing benchmark data may be made accessible to a web browser via a web services or cloud API. Access is allowed via a secure web portal. The web portal may be secured, for example, with a username and password. Access may include receiving a request from the user, where the request further includes one or more filters or criteria. The user can view and interact with the benchmark data. Results based on filter criteria may be displayed to the user. Exemplary filter criteria include surgical location type, procedure list, surgical volume. Results returned by the system may be associated with, for example, one or more of the following three categories.

A first category of results includes recommendations related to supplies and instruments associated with a procedure. The first category may also include an indication of the amount of the supplies and instruments are associated with a procedure, as well as an additional buffer amount of the supplies and instruments desired. The additional buffer amount of the supplies and instruments protects against failure to have enough of the supplies and instruments for the procedure if one of the supplies or instruments is damaged, lost, undercounted, or otherwise unusable during the procedure. Without the additional buffer amount, the tray would, at best, have exactly the number of supplies and instruments required for the procedure and, at worst, have less than the number of supplies and instruments required for the procedure. While having exactly the required number of supplies and instruments for the procedure is most efficient (e.g., least expensive because it minimizes the number of supplies and instruments per tray), a risk is that a procedure may be unable to be performed if more supplies and instruments are required that were estimated and/or any of the supplies or instruments are damaged or undercounted. Moreover, this risk is likely only to be discovered during the procedure and have significant negative impact on the patient.

A second category of results includes information related to the mix of instruments associated with a procedure and recommendations for optimal tray configuration. For example, for a heart procedure, three scalpels, two clamps, and ten gauze pads may be associated with the procedure. An optimal tray configuration may include this mixture of instrument and supplies. These procedure mix instrument usage and optimal tray configuration recommendations may be displayed in a manner conducive to a user assembling a tray based on the recommended tray configuration.

A third category of results includes projections of various metrics. The projections estimate, predict, extrapolate, or otherwise project a metric into a future time period. For example, a projected metric may include a total number of bandages used across all procedures per year. These instrument tray inventory projections based on procedure mix, annual volume, sterilization turnaround time, etc. For example, in a scenario where ten bandages are associated with a first procedure and three bandages are associated with a second procedure, the projected number of bandages used per year across both procedures may be based on both the per-procedure usage of bandages and the number of times each procedure is estimated to be performed per year. Continuing the example above, in a scenario where the first procedure is estimated to be performed ten times per year and the second procedure is estimated to be performed five times per year, the projected number of bandages used per year across both procedures would be 115 bandages (i.e., (10 bandages×10 procedures)+(3 bandages×5 procedures)=115 bandages).

Database and machine learning algorithm 302 may communicate the proposed tray configurations and data-based associations to predictive model 304.

### Predictive Model

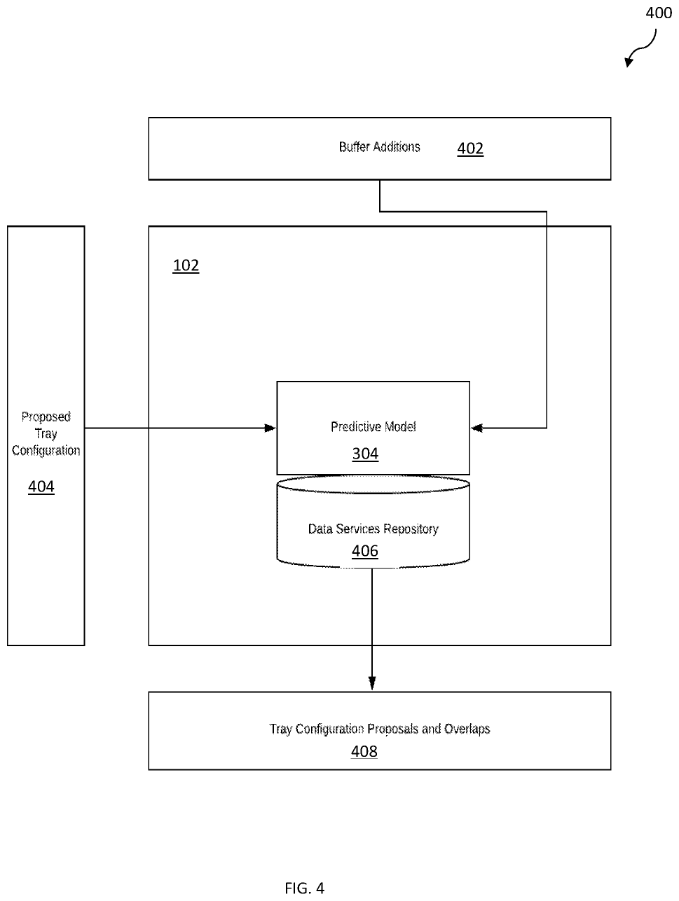

FIG. 4 is a functional block diagram illustrating a machine learning model and instrument recommendation algorithm for tray configurations in accordance with at least one embodiment of the present invention. Referring to FIG. 4, process 400 begins when predictive model 304 receives buffer additions 402 and proposed tray configuration 404. Predictive model 304 may be associated with, and communicatively coupled to, data services repository 406. The output of predictive model 304 includes tray configuration proposals and overlaps 408.

For example, once the proposed tray is defined and predictive model 304 is run, the user may create an audit sheet to be used within the operating room to validate the tray configuration. Surgical staff will pull the newly defined tray configuration instruments from the existing trays on the sterile field at the beginning of the case. At the end of the case, a count will be performed to document the actual usage from the proposed tray. Surgical staff will then provide feedback to add required instruments that are not often used but may be required for patient safety or other reasons. Updates from the audit will be reflected in the proposed tray configurations. After the required number of audits are complete and revisions performed, the new tray is ready for implementation and assignment to preference cards. During the tray rationalization process, a further predictive model utilizing related instruments and tray associations is run to determine based on the Specialty, Location, Surgeon, Procedure and CPT code(s) which instruments are likely needed in addition to the instruments that have actual usage collected in counts. Predictive model 304 identifies specific instruments that would need to be added and the quantity needed.

Predictive model 304 utilizes other approved tray configurations as well as the Data Services repository 406 for standardized tray configurations. Predictive model 304 is executed and recommendations are made within the application on the tray configuration section. Recommendations are accepted by the user before they are applied to the tray configuration.

### Tray Consolidation Engine

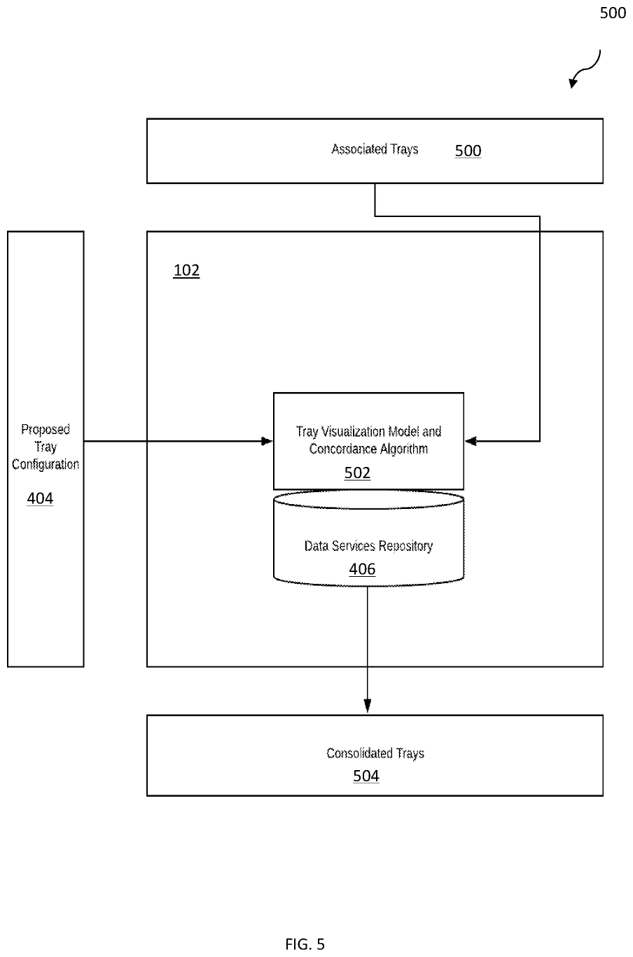

Proposed tray configurations can be used to replace more than one tray that already exists. Within the rationalization process, the software allows a user to visualize multiple trays in comparison to the proposed trays to see instruments that are on existing trays and allow addition of them to the proposed tray. The visualization allows users to see the concordance of instruments across all trays selected with the proposed tray. Users create a proposed tray configuration that pulls instruments from multiple trays onto one proposed tray by choosing the overlap percentage required for trays associated with the same preference card. The concordance engine then produces the amount of overlap existing between consolidated tray and all the other trays on the associated preference cards as depicted in FIG. 5.

FIG. 5 is a functional block diagram illustrating a consolidation engine configured to reduce the number of trays sharing similar instruments and reduce tray instance proliferation in accordance with at least one embodiment of the present invention. Referring to FIG. 5, process 500 begins when tray visualization model and concordance algorithm 502 receives associated trays 500 and proposed tray configuration 404 information. Tray visualization model and concordance algorithm 502 may be associated with, and communicatively coupled to, data services repository 406. The output of tray visualization model and concordance algorithm 502 includes consolidated trays 504.

For example, if one hundred percent coverage exists, then a simple transfer from one tray to another is done. If less than one hundred percent coverage exists, the trays are presented in descending order of overlap and the highest overlap percentage tray is recommended as the first transfer candidate. Once validated, the transfer takes place and the impact on instrument inventory is visible. After the transfer takes place, the preference cards are updated, This creates a closed feedback loop for the new consolidated trays for the relevant procedures.

### Synchronization with Preference Cards and Pick Lists

Once complete, the user can view all preference cards that use the existing trays consolidated on the new tray to see if one hundred percent instrument coverage was achieved for cards selected. The software then uses the card assignment process to output a change request for existing preference cards or pick lists to change the existing trays to the newly proposed tray. Users can pick a specialty, surgeon, location or CPT code(s) to show the instrument coverage for assigning a new tray to existing preference cards. Users can view all preference cards they select and for those cards that have one hundred percent coverage and a case count above their minimum threshold they can select the new tray to replace the existing trays on the cards.

A report is generated by the product that shows the Card, Surgeon, Specialty, location and the old tray(s) and new replacement tray. This is used to update external systems that manage the supply chain portions of the surgical process.

### Benchmark Web Service Access

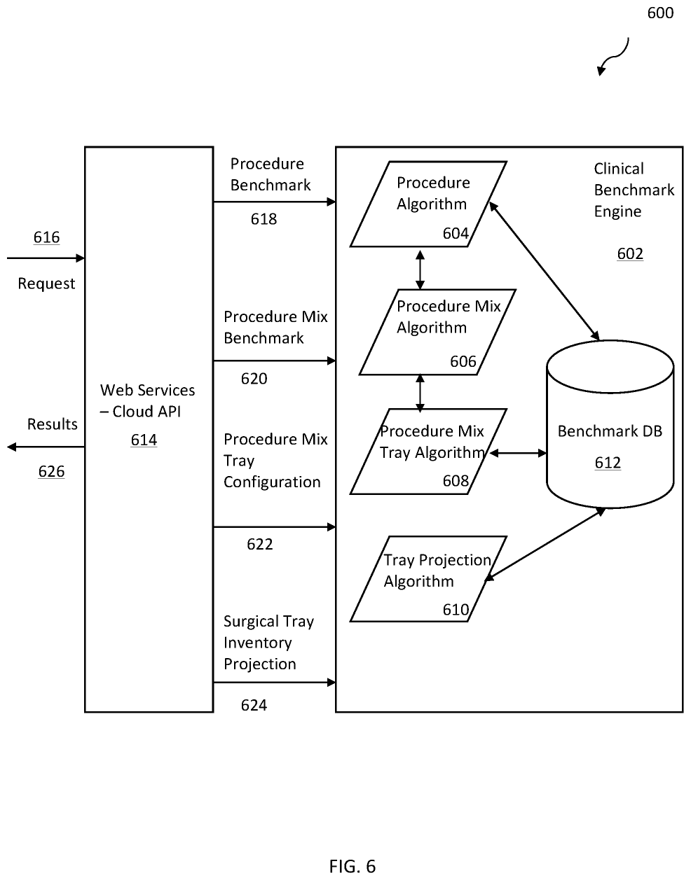

Benchmarks can include usage data, buffer data, and recommendations. Benchmarks are based on clinical reviews and are stored within a database. Benchmarks can be accessed via the web services interface as depicted in FIG. 6 and filtered based on filter criteria. Authenticated users have four different methods for interacting with the benchmarks that return differing data sets based on each request. Each request allows filter criteria to limit the data returned from the benchmarks. The filter criteria include, but are not limited to, a surgical location type, an identity of procedure(s), and a service line.

FIG. 6 is a functional block diagram illustrating the use of a web services cloud API interface for a clinical benchmark database in accordance with at least one embodiment of the present invention. Referring to FIG. 6, system 600 includes clinical benchmark engine 602. Clinical benchmark engine 502 includes: procedure algorithm 604, procedure mix algorithm 606, procedure mix tray algorithm 608, and tray projection algorithm 610. Each of algorithms 604, 606, 608, and 610 may be associated with, and communicatively coupled to, benchmark database 612 for storing data.

Clinical benchmark engine 502 may be accessed via web services cloud API interface 614. For example, a request 616 received by web services cloud API interface 614 may be routed to clinical benchmark engine 502 as one of, for example, procedure benchmark 618, procedure mix benchmark 620, procedure mix tray configuration 622, or surgical tray inventory projection 624. Once processed by clinical benchmark engine 502, results 626 may be returned to the user via web services cloud API interface 614.

In one embodiment, the interface request includes procedure benchmark by surgical location type. A response to this request returns average instrument usage, buffer instrument quantity and recommended quantity, average supply usage, supply buffer and supply recommendation.

In one embodiment, the interface request includes procedure mix benchmark by surgical location type and service line. A response to this request returns average instrument usage, buffer instrument quantity and recommended quantity for multiple procedures requested averaged together.

In one embodiment, the interface request includes procedure mix tray configuration by surgical location type and service line. A response to this request returns tray name, instrument name, instrument quantity for the trays that are defined within benchmarks for the procedure mix submitted.

In one embodiment, the interface request includes surgical tray inventory projection by surgical location type, service line and procedure mix. A response to this request returns the project quantity of instrument trays by location that are needed in inventory to support surgical volume for the tray. Request requires historical volume of sterilization by tray per year, time to sterilize each tray, par stock standard by location, and number copies of each tray.

A benefit of the benchmark data includes the ability to determine what is required for a specific case via being imbedded in other systems. Another benefit of benchmark data includes the ability to assess which procedures can be improved. For example, potential for improvement can be based on comparisons and similarities between procedures. Another benefit of benchmark data includes providing a planning process for hospitals that is empirically based on surgical volume and the needs for trays. Another benefit of benchmark data includes providing a process for defining new tray definitions, which may streamline tray configuration.

The subject matter described herein includes a method performed by a computer system that executes non-transitory computer-readable instructions stored on a computer readable storage medium. The method includes data collection for the analysis and rationalization of instrument trays within hospitals, health systems and outpatient surgery centers. The method includes collecting and storing data on instrument usage. In one embodiment, a value of instruments used may be collected. For example, it may be important to track the usage of a high value or expensive instrument in greater detail than the usage of low value or less expensive instruments. In another embodiment, a volume and/or a frequency of instrument tray usage may be collected and stored in a database. For example, a first instrument tray may be used five times per day an average of two days per week while a second instrument tray may be used once per month for every month. Further, usage of the first instrument tray may be greater during a first month and less in a second month. While the average across the first and second months may be determined and stored, it may also be important to determine and store the difference in usage of the first tray between the first and second months, to predict and plan for more usage of the first tray during the first month.

The method further includes collecting and storing: data on preference card instrumentation, equipment, and disposables; existing tray definitions and configurations in a database; surgeon, specialty, and procedure data; and procedure, preference card and associated tray data. The method further includes determining, by a computer, cases requiring data collection and setting flags to provide trigger events indicating cases that need to be counted by the data collection team. The method further includes determining, by computer analysis, the instruments that can be removed from the selected trays due to non-usage or low usage. The method further includes determining, by a computer algorithm, the added buffer and designating a proposed new tray configuration as complete after determination of the added buffer. The method further includes determining, by computer algorithm, other trays or cards that are associated with the proposed new tray configurations. The other associated trays or cards can be reconfigured to reduce instrument usage or proliferation. The method further includes determining, by the consolidation engine, trays associated with particular preference cards. This allows the number of trays to be reduced. For example, multiple trays may be consolidated into one tray. In another example, multiple trays may be consolidated into a lesser number of trays than previous tray configuration(s).

In one embodiment, related elements are associated with each other in the data collection model. This provides visibility into both targeted tray configurations and associated trays that include related instruments used in other procedures, related instruments used by other surgeons, and related preference cards. For example, a particular tray or tray configuration may include a first instrument, such as a scalpel. The scalpel may be associated with a second instrument, such as a spreader, because all procedures performed with the first instrument also require use of the second instrument. As a result, trays, or tray configurations, that include the first instrument may automatically also include the second instrument based on this association stored in the database as defined by the data collection model. It is appreciated that each element may be associated with, or determined to be related to, any number of other elements. For example, a first instrument may be related to ten other instruments while a second instrument may be associated with no other instruments.

In one embodiment, a machine learning algorithm is configured to determine (e.g., suggest or propose to the user) new tray configurations based on the stored database associations and relationships between procedures, preference cards, and common instruments.

In one embodiment, a predictive model proposes new tray configurations resulting from the machine learning algorithm.

In one embodiment, a consolidation engine determines all existing trays associated with targeted preference cards and procedures and consolidates the existing trays into fewer trays. The consolidation may be configured to optimize for a desired criteria. In one embodiment, the criteria to be optimized may be the number of trays. As a result, the consolidation engine may minimize the number of trays, thereby reducing instrument inventory level and re-processing and sterilization costs. In other embodiments, the consolidation engine may be configured to optimize the average or total number of instruments on the consolidated set of trays, the average cost of each tray, or other factors.

In one embodiment, a web services portal allows interaction with surgical benchmarks to return results of specific queries about instruments (usage, buffers and recommended quantities), supplies (usage and recommendations), tray configurations and tray inventory projects based on filter criteria within the web service interface.

Machine learning (ML) is the use of computer algorithms that can improve automatically through experience and by the use of data. Machine learning algorithms build a model based on sample data, known as training data, to make predictions or decisions without being explicitly programmed to do so. Machine learning algorithms are used where it is unfeasible to develop conventional algorithms to perform the needed tasks.

In certain embodiments, instead of or in addition to performing the functions described herein manually, the system may perform some or all of the functions using machine learning or artificial intelligence. Thus, in certain embodiments, machine learning-enabled software relies on unsupervised and/or supervised learning processes to perform the functions described herein in place of a human user.

Machine learning may include identifying one or more data sources and extracting data from the identified data sources. Instead of or in addition to transforming the data into a rigid, structured format, in which certain metadata or other information associated with the data and/or the data sources may be lost, incorrect transformations may be made, or the like, machine learning-based software may load the data in an unstructured format and automatically determine relationships between the data. Machine learning-based software may identify relationships between data in an unstructured format, assemble the data into a structured format, evaluate the correctness of the identified relationships and assembled data, and/or provide machine learning functions to a user based on the extracted and loaded data, and/or evaluate the predictive performance of the machine learning functions (e.g., “learn” from the data).

In certain embodiments, machine learning-based software assembles data into an organized format using one or more unsupervised learning techniques. Unsupervised learning techniques can identify relationship between data elements in an unstructured format.

In certain embodiments, machine learning-based software can use the organized data derived from the unsupervised learning techniques in supervised learning methods to respond to analysis requests and to provide machine learning results, such as a classification, a confidence metric, an inferred function, a regression function, an answer, a prediction, a recognized pattern, a rule, a recommendation, or other results. Supervised machine learning, as used herein, comprises one or more modules, computer executable program code, logic hardware, and/or other entities configured to learn from or train on input data, and to apply the learning or training to provide results or analysis for subsequent data.

Machine learning-based software may include a model generator, a training data module, a model processor, a model memory, and a communication device. Machine learning-based software may be configured to create prediction models based on the training data. In some embodiments, machine learning-based software may generate decision trees. For example, machine learning-based software may generate nodes, splits, and branches in a decision tree. Machine learning-based software may also calculate coefficients and hyper parameters of a decision tree based on the training data set. In other embodiments, machine learning-based software may use Bayesian algorithms or clustering algorithms to generate predicting models. In yet other embodiments, machine learning-based software may use association rule mining, artificial neural networks, and/or deep learning algorithms to develop models. In some embodiments, to improve the efficiency of the model generation, machine learning-based software may utilize hardware optimized for machine learning functions, such as an FPGA.

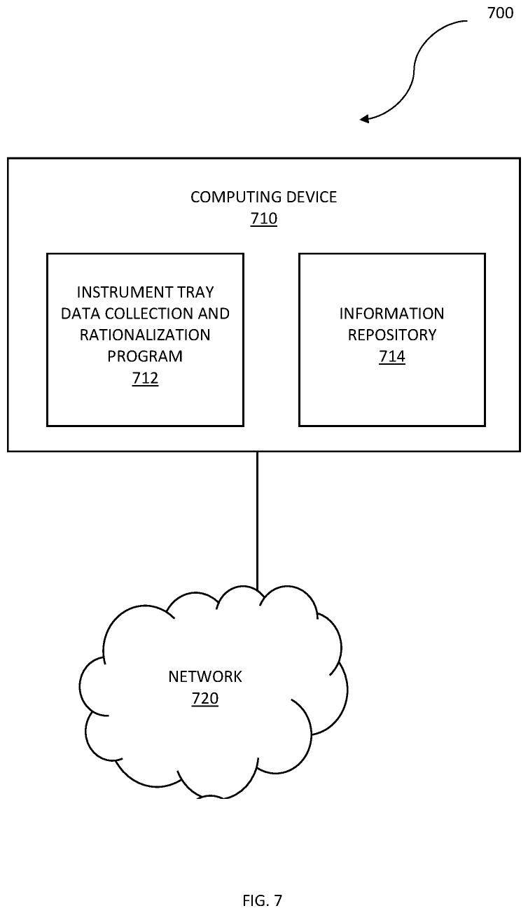

FIG. 7 is a functional block diagram illustrating a distributed data processing environment, generally designated 700, suitable for operation of instrument tray data collection and rationalization program 712 in accordance with at least one embodiment of the present invention. The term “distributed” as used herein describes a computer system that includes multiple, physically distinct devices that operate together as a single computer system. FIG. 7 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 700 includes computing device 710 connected to network 720. Network 720 can be, for example, a telecommunications network, a local area network (LAN), a wide area network (WAN), such as the Internet, or a combination of the three, and can include wired, wireless, or fiber optic connections. Network 720 can include one or more wired and/or wireless networks that are capable of receiving and transmitting data, voice, and/or video signals, including multimedia signals that include voice, data, and video information. In general, network 720 can be any combination of connections and protocols that will support communications between computing device 710 and other computing devices (not shown) within distributed data processing environment 700.

Computing device 710 can be a standalone computing device, a management server, a web server, a mobile computing device, or any other electronic device or computing system capable of receiving, sending, and processing data. In an embodiment, computing device 710 can be a smart phone, a laptop computer, a tablet computer, a netbook computer, a personal computer (PC), a desktop computer, a personal digital assistant (PDA), or any programmable electronic device capable of communicating with other computing devices (not shown) within distributed data processing environment 700 via network 720. In another embodiment, computing device 710 can represent a server computing system utilizing multiple computers as a server system, such as in a cloud computing environment. In yet another embodiment, computing device 710 represents a computing system utilizing clustered computers and components (e.g., database server computers, application server computers, etc.) that act as a single pool of seamless resources when accessed within distributed data processing environment 100.

In an embodiment, computing device 710 includes instrument tray data collection and rationalization program 712. In an embodiment, instrument tray data collection and rationalization program 712 is a program, application, or subprogram of a larger program for instrument tray data collection and rationalization tracking. In an alternative embodiment, instrument tray data collection and rationalization program 712 may be located on any other device accessible by computing device 710 via network 720.

In an embodiment, computing device 710 includes information repository 114. In an embodiment, information repository 714 may be managed by instrument tray data collection and rationalization program 712. In an alternate embodiment, information repository 714 may be managed by the operating system of the device, alone, or together with, instrument tray data collection and rationalization program 712. Information repository 714 is a data repository that can store, gather, compare, and/or combine information. In some embodiments, information repository 714 is located externally to computing device 710 and accessed through a communication network, such as network 120. In some embodiments, information repository 714 is stored on computing device 710. In some embodiments, information repository 714 may reside on another computing device (not shown), provided that information repository 714 is accessible by computing device 710. Information repository 714 includes, but is not limited to, data that is received by instrument tray data collection and rationalization program 712 from one or more sources, and data that is created by instrument tray data collection and rationalization program 712.

Information repository 714 may be implemented using any volatile or non-volatile storage media for storing information, as known in the art. For example, information repository 714 may be implemented with a tape library, optical library, one or more independent hard disk drives, multiple hard disk drives in a redundant array of independent disks (RAID), solid-state drives (SSD), or random-access memory (RAM). Similarly, information repository 714 may be implemented with any suitable storage architecture known in the art, such as a relational database, an object-oriented database, or one or more tables.

Information repository 714 includes a database. In an embodiment, instrument tray data collection and rationalization program 712 creates a database, if it does not already exist, for a user of the system to store data. The database allows instrument tray data collection and rationalization program 712 to provide information requested by instrument tray data collection and rationalization program 712.

The system disclosed herein may be implemented as a client/server type architecture but may also be implemented using other architectures, such as cloud computing, software as a service model (SaaS), a mainframe/terminal model, a stand-alone computer model, a plurality of non-transitory lines of code on a computer readable medium that can be loaded onto a computer system, a plurality of non-transitory lines of code downloadable to a computer and the like which are within the scope of the disclosure.

The system may be implemented as one or more computing devices that connect to, communicate with and/or exchange data over a link that interact with each other. Each computing device may be a processing unit-based device with sufficient processing power, memory/storage and connectivity/communications capabilities to connect to and interact with the system. For example, each computing device may be an Apple iPhone or iPad product, a Blackberry or Nokia product, a mobile product that executes the Android operating system, a personal computer, a tablet computer, a laptop computer and the like and the system is not limited to operate with any particular computing device. The link may be any wired or wireless communications link that allows the one or more computing devices and the system to communicate with each other. In one example, the link may be a combination of wireless digital data networks that connect to the computing devices and the Internet. The system may be implemented as one or more server computers (all located at one geographic location or in disparate locations) that execute a plurality of lines of non-transitory computer code to implement the functions and operations of the system as described herein. Alternatively, the system may be implemented as a hardware unit in which the functions and operations of the back-end system are programmed into a hardware system. In one implementation, the one or more server computers may use Intel® processors, run the Linux operating system, and execute Java, Ruby, Regular Expression, Flex 4.0, SQL etc.

In some embodiments, each computing device may further comprise a display and a browser application so that the display can display information generated by the system. The browser application may be a plurality of non-transitory lines of computer code executed by a processing unit of the computing device. Each computing device may also have the usual components of a computing device such as one or more processing units, memory, permanent storage, wireless/wired communication circuitry, an operating system, etc.

The system may further comprise a server (that may be software based or hardware based) that allows each computing device to connect to and interact with the system such as sending information and receiving information from the computing devices that is executed by one or more processing units. The system may further comprise software- or hardware-based modules and database(s) for processing and storing content associated with the system, metadata generated by the system for each piece of content, user preferences, and the like.

In one embodiment, the system includes one or more processors, server, clients, data storage devices, and non-transitory computer readable instructions that, when executed by a processor, cause a device to perform one or more functions. It is appreciated that the functions described herein may be performed by a single device or may be distributed across multiple devices.

When a user interacts with the system, the user may use a frontend client application. The client application may include a graphical user interface that allows the user to select one or more digital files. The client application may communicate with a backend cloud component using an application programming interface (API) comprising a set of definitions and protocols for building and integrating application software. As used herein, an API is a connection between computers or between computer programs that is a type of software interface, offering a service to other pieces of software. A document or standard that describes how to build or use such a connection or interface is called an API specification. A computer system that meets this standard is said to implement or expose an API. The term API may refer either to the specification or to the implementation.

Software-as-a-service (SaaS) is a software licensing and delivery model in which software is licensed on a subscription basis and is centrally hosted. SaaS is typically accessed by users using a thin client, e.g., via a web browser. SaaS is considered part of the nomenclature of cloud computing.

Many SaaS solutions are based on a multitenant architecture. With this model, a single version of the application, with a single configuration (hardware, network, operating system), is used for all customers (“tenants”). To support scalability, the application is installed on multiple machines (called horizontal scaling). The term “software multitenancy” refers to a software architecture in which a single instance of software runs on a server and serves multiple tenants. Systems designed in such manner are often called shared (in contrast to dedicated or isolated). A tenant is a group of users who share a common access with specific privileges to the software instance. With a multitenant architecture, a software application is designed to provide every tenant a dedicated share of the instance—including its data, configuration, user management, tenant individual functionality and non-functional properties.

The backend cloud component described herein may also be referred to as a SaaS component. One or more tenants which may communicate with the SaaS component via a communications network, such as the Internet. The SaaS component may be logically divided into one or more layers, each layer providing separate functionality and being capable of communicating with one or more other layers.

Cloud storage may store or manage information using a public or private cloud. Cloud storage is a model of computer data storage in which the digital data is stored in logical pools. The physical storage spans multiple servers (sometimes in multiple locations), and the physical environment is typically owned and managed by a hosting company. Cloud storage providers are responsible for keeping the data available and accessible, and the physical environment protected and running. People and/or organizations buy or lease storage capacity from the providers to store user, organization, or application data. Cloud storage services may be accessed through a co-located cloud computing service, a web service API, or by applications that utilize the API.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method comprising instructions stored on a non-transitory computer-readable storage medium and executed on a computing device provided with a hardware processor and a memory for collection and rationalization of instrument trays within a healthcare environment, the method comprising:
determining and storing equipment information related to equipment inventory;
determining and storing disposable article information related to disposable article inventory;
determining and storing preference card information related to surgical equipment and disposable articles used during a procedure;
determining a tray configuration for a procedure based on the preference card information, the equipment information, and the disposable article information,
wherein the tray configuration identifies articles to be placed within an instrument tray;
determining instrument usage information for articles placed within the instrument tray during the procedure;
determining, based on the instrument usage information, whether the articles placed within the instrument tray are used at a frequency below a predetermined threshold; and
in response to determining that the articles placed within the instrument tray are used at a frequency below the predetermined threshold, updating the preference card information and the tray configuration for the procedure.

2. The computer-implemented method of claim 1, further comprising receiving information related to surgeons, specialty, and procedures.

3. The computer-implemented method of claim 1, further comprising generating an alert in response to determining that a tray configuration is incomplete for a subsequent procedure.

4. The computer-implemented method of claim 3, further comprising sending the alert to the user.

5. The computer-implemented method of claim 3, further comprising automatically initiating data collection for the subsequent procedure in response to the alert.

6. The computer-implemented method of claim 1, further comprising determining a buffer addition for a tray configuration.

7. The method of claim 1, further comprising determining other tray configurations related to the updated tray configuration and updating the other tray configurations based on the updated tray configuration.

8. The computer-implemented method of claim 1, further comprising determining that other tray configurations are associated with the updated preference cards and updating the other tray configurations based on the updated preference cards.

9. The computer-implemented method of claim 1, further comprising reducing a number of instrument trays based on the updated preference cards.

10. The computer-implemented method of claim 1, further comprising:
displaying a user interface on a remote computer;
receiving queries executed by a user; and
providing search results in response to the received queries.

11. A system for collection and rationalization of instrument trays within a healthcare environment as software a service (SaaS), the system comprising:
a computer communicatively coupled to a network, at least one S aaS provider, and at least one SaaS user, and wherein the computer is configured to:
determine and store equipment information related to equipment inventory;
determine and store disposable article information related to disposable article inventory;
determine and store preference card information related to surgical equipment and disposable articles used during a procedure;
determine a tray configuration for a procedure based on the preference card information, the equipment information, and the disposable article information,
wherein the tray configuration identifies articles to be placed within an instrument tray;
determine instrument usage information for articles placed within the instrument tray during the procedure;
determine, based on the instrument usage information, whether the articles placed within the instrument tray are used at a frequency below a predetermined threshold; and
in response to determining that the articles placed within the instrument tray are used at a frequency below the predetermined threshold, update the preference card information and the tray configuration for the procedure.
